(19) United States
(12) Patent Application Publication
(10) Pub. No.: US 2026/0077008 A1
(43) Pub. Date: Mar. 19, 2026

(54) COMPOSITION FOR PROMOTING COGNITIVE FUNCTIONS AND METHODS OF MAKING AND USING THE SAME

(71) Applicant: Michael Nakamura, Laupahoehoe, HI (US)

(72) Inventor: Michael Nakamura, Laupahoehoe, HI (US)

(21) Appl. No.: 19/329,162

(22) Filed: Sep. 15, 2025

Related U.S. Application Data
(60) Provisional application No. 63/694,814, filed on Sep. 14, 2024.

(51) Int. Cl. A61K 36/185

(52) U.S. Cl. CPC A61K 36/185; A61K 2236/17; A61K 2236/31

(57) ABSTRACT

Compositions of the present invention are conducive to the promotion of cognitive functions. Specifically, the compositions of the present invention comprise a formulation derived from the fruit, leaf, and stem of Pipturis albidis, also known as Mamaki. Specifically, the formulation comprises a therapeutically effective amount of an active composition consisting of greater than about 5% material obtained from the dried fruit of Pipturis albidis with the balance of the active composition comprising material obtained from the leaves and/or the stem thereof. Optionally, one or more excipient, or inactive ingredients, may be present.

### TECHNICAL FIELD

The present invention relates to compositions conducive to the promotion of cognitive functions. Specifically, the compositions of the present invention comprise a formulation derived from the fruit, leaf, and stem of Pipturis albidis, also known as Mamaki. Specifically, the formulation comprises a therapeutically effective amount of an active composition consisting of greater than about 5% material obtained from the dried fruit of Pipturis albidis with the balance of the active composition comprising material obtained from the leaves and/or the stem thereof. Optionally, one or more excipient, or inactive ingredients, may be present.

### BACKGROUND

The Mamaki plant, known by its taxonomy as Pipturis albidis, is generally known in Hawaiian folklore for a host of benefits, including regulating blood glucose levels, blood pressure, and cholesterol levels, relieving stress and fatigue; and reducing inflammation. Subsequent studies on the Mamaki plant indicate that the plant contains a significant amount and number of polyphenols having anti-inflammatory and anti-oxidant activities.

As the human brain ages, cognition declines. In some cases, common diseases, such as, for example, Alzheimer's disease, dementia, or other like conditions can lead to significant cognitive decline in relatively short time periods. Dementia and other forms of significant cognitive decline are major factors in both quality and quantity of life as humans age.

A need exists for an improved composition for promoting cognition in humans and/or animals. Specifically, a need exists for an improved composition for improving memory and recall when ingested. More specifically, a need exists for an improved composition for promoting overall cognitive function, improving brain health, and aiding in the prevention of dementia.

Moreover, a need exists for an improved composition that is plant based. Specifically, a need exists for an improved composition derived from Pipturis albidis, also known as the Mamaki plant. More specifically, a need exists for an improved composition derived from Pipturis albidis that improves cognition, promotes brain health, and aids in the prevention of dementia.

Further, a need exists for improved compositions and methods of making and using the same that may be utilized by humans as well as other creatures, such as pets. Specifically, a need exists for improved compositions and methods of making and using the same that may be in the form of beverages, foods, supplements, snacks, pet treats and pet food, tinctures, or in other like forms that may be easily administered to a user thereof.

### SUMMARY OF THE INVENTION

The present invention relates to compositions conducive to the promotion of cognitive functions. Specifically, the compositions of the present invention comprise a formulation derived from the fruit, leaf, and stem of Pipturis albidis, also known as Mamaki. Specifically, the formulation comprises a therapeutically effective amount of an active composition consisting of greater than about 5% material obtained from the dried fruit of Pipturis albidis with the balance of the active composition comprising material obtained from the leaves and/or the stem thereof. Optionally, one or more excipient, or inactive ingredients, may be present.

To this end, in an embodiment of the present invention, a dietary composition formulated in a therapeutic effective amount to promote cognition in humans or animals is provided. The dietary composition comprises a therapeutically effective amount of a composition consisting of greater than 5% material obtained from the dried fruit of the Pipturis albidis plant; the balance of the therapeutically effective amount of the composition comprises material derived from the leaves and/or stem of the Pipturis albidis plant. The material derived from the dried fruit of Pipturis albidis and the material derived from the leaves and/or stem of Pipturis albidis is in the form of a powdered extract.

In an embodiment, the dietary composition is in the form of a pill, a capsule, a tablet, a softgel, a lozenge, a powder, a liquid, a food, a beverage, a gummy, or an orally-dissolving film strip.

In an alternate embodiment of the present invention, a dietary composition formulated in a therapeutic effective amount to promote cognition in humans or animals is provided. The dietary composition consists of: a therapeutically effective amount of a composition consisting of greater than about 5% material obtained from the dried fruit of the Pipturis albidis plant; the balance of the therapeutically effective amount of the composition comprising material derived from the leaves, the stem, or the leaves and the stem of the Pipturis albidis plant; and optionally, one or more excipients.

In yet another alternate embodiment of the present invention, a method of promoting cognition in humans or animals is provided. The method comprises the steps of: providing a dietary composition formulated in a therapeutic effective amount to promote cognition in humans or animals comprising a therapeutically effective amount of a composition consisting of greater than about 5% material obtained from the dried fruit of the Pipturis albidis plant, the balance of the therapeutically effective amount of the composition comprising material derived from the leaves, the stem, or the leaves and the stem of the Pipturis albidis plant; and optionally, one or more excipients; and administering to a human or animal the dietary composition.

It is, therefore, an advantage and objective of the present invention to provide improved compositions and methods of making and using the same for promoting cognition.

Specifically, it is an advantage and objective of the present invention to provide improved compositions for improving memory and recall when ingested.

More specifically, it is an advantage and objective of the present invention to provide improved compositions for promoting overall cognitive function, improving brain health, and aiding in the prevention of dementia.

Moreover, it is an advantage and objective of the present invention to provide improved compositions that are natural and plant-based.

Specifically, it is an advantage and objective of the present invention to provide improved compositions derived from Pipturis albidis, also known as the Mamaki plant.

More specifically, it is an advantage and objective of the present invention to provide improved compositions derived from Pipturis albidis that improve cognition, promote brain health, and aid in the prevention of dementia.

Further, it is an advantage and objective of the present invention to provide improved compositions and methods of making and using the same that may be utilized by humans as well as other creatures, such as pets.

Specifically, it is an advantage and objective of the present invention to provide improved compositions and methods of making and using the same that may be in the form of beverages, foods, supplements, snacks, pet treats and pet food, tinctures, or in other like forms that may be easily administered to a user thereof.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention relates to compositions conducive to the promotion of cognitive functions. Specifically, the compositions of the present invention comprise a formulation derived from the fruit, leaf, and stem of Pipturis albidis, also known as Mamaki. Specifically, the formulation comprises a therapeutically effective amount of an active composition consisting of greater than about 5% material obtained from the dried fruit of Pipturis albidis with the balance of the active composition comprising material obtained from the leaves and/or the stem thereof. Optionally, one or more excipient, or inactive ingredients, may be present.

For the purposes of the present invention, the term “improved cognition” is defined as having better memory and recall when accomplishing tasks. As such, “improved cognition” can be measured by objective and subjective means. The compositions of the present invention, described herein, are designed to target biological components promoting and supporting brain cognition in humans and/or animals.

The composition of the present invention comprises various amounts of extracts or ingredients (i.e., purified compounds derived from plants) that are derived from the dried fruit and the leaves and/or stem of Pipturis albidis, combined together into a blend that is designed to promote improved cognition.

Preferably, dietary compositions formulated herein may be in a therapeutic effective amount to promote cognition in humans or animals. The dietary composition comprises a therapeutically effective amount of a composition consisting of greater than 5% material obtained from the dried fruit of the Pipturis albidis plant; the balance of the therapeutically effective amount of the composition comprises material derived from the leaves and/or stem of the Pipturis albidis plant. The material derived from the dried fruit of Pipturis albidis and the material derived from the leaves and/or stem of Pipturis albidis may preferably be in the form of a powdered extract.

The compositions of the present invention may further comprise excipients, which may include one or more emulsifiers, sweeteners, bulking agents, mouthfeel and/or flavor agents, and other agents or ingredients suitable for food and/or beverage compositions. It should be noted that the amounts and percentages preferred in the present invention are measured against the total of the blend of the active ingredients listed above without regard to additional ingredients are provided in the compositions described herein

The dietary compositions described herein may be in the form of a pill, a capsule, a tablet, a softgel, a lozenge, a powder, a liquid, a food, a beverage, a gummy, or an orally-dissolving film strip.

Cognitive tests administered to humans before and after taking formulations of the present invention led to marked improvements in cognitive functions. Specifically, in a carefully controlled study with small test groups before administration and after seven days of administration of the formulations described herein, one hundred percent of respondents showed improvement with cognition.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

What is claimed is:

1. A dietary composition formulated in a therapeutic effective amount to promote cognition in
humans or animals comprising:
a therapeutically effective amount of a composition consisting of greater than about 5% material obtained from the dried fruit of the Pipturis albidis plant;
the balance of the therapeutically effective amount of the composition comprising material derived from the leaves, the stem, or the leaves and the stem of the Pipturis albidis plant; and
optionally, one or more excipients.

2. The dietary composition of claim 1 wherein the material derived from the dried fruit of Pipturis albidis and the material derived from the leaves, the stem, or the leaves and the stem of Pipturis albidis is in the form of a powdered extract.

3. The dietary composition of claim 1, wherein the dietary composition is in the form of a pill, a capsule, a tablet, a softgel, a lozenge, a powder, a liquid, a food, a beverage, a gummy, or an orally-dissolving film strip.

4. A dietary composition formulated in a therapeutic effective amount to promote cognition in humans or animals consisting of:
a therapeutically effective amount of a composition consisting of greater than about 5% material obtained from the dried fruit of the Pipturis albidis plant;
the balance of the therapeutically effective amount of the composition comprising material derived from the leaves, the stem, or the leaves and the stem of the Pipturis albidis plant; and
optionally, one or more excipients.

5. The dietary composition of claim 4 wherein the material derived from the dried fruit of Pipturis albidis and the material derived from the leaves, the stem, or the leaves and the stem of Pipturis albidis is in the form of a powdered extract.

6. The dietary composition of claim 4, wherein the dietary composition is in the form of a pill, a capsule, a tablet, a softgel, a lozenge, a powder, a liquid, a food, a beverage, a gummy, or an orally-dissolving film strip.

7. A method of promoting cognition in humans or animal comprising the steps of:
providing a dietary composition formulated in a therapeutic effective amount to promote cognition in humans or animals comprising a therapeutically effective amount of a composition consisting of greater than about 5% material obtained from the dried fruit of the Pipturis albidis plant, the balance of the therapeutically effective amount of the composition comprising material derived from the leaves, the stem, or the leaves and the stem of the Pipturis albidis plant; and optionally, one or more excipients; and
administering to a human or animal the dietary composition.

8. The method of claim 7 wherein the material derived from the dried fruit of Pipturis albidis and the material derived from the leaves, the stem, or the leaves and the stem of Pipturis albidis is in the form of a powdered extract.

9. The method of claim 7, wherein the dietary composition is in the form of a pill, a capsule, a tablet, a softgel, a lozenge, a powder, a liquid, a food, a beverage, a gummy, or an orally-dissolving film strip.
